(19) United States
(12) Patent Application Publication
(10) Pub. No.: US 2016/0317066 A1
(43) Pub. Date: Nov. 3, 2016

(54) SPASTICITY QUANTIFICATION DEVICE

(71) Applicant: Accelerated Rehabilitation Technologies, LLC, St. Louis, MO (US)

(72) Inventors: Tony Ying-Wen Wang, St. Louis, MO (US); Charles Yong-Chao Wu, New York, NY (US); Olivia Mae Sutton, Barrington, IL (US)

(21) Appl. No.: 15/143,544

(22) Filed: Jul. 18, 2016

Related U.S. Application Data
(60) Provisional application No. 62/155,669, filed on May 1, 2015.

(51) Int. Cl. A61B 5/11; A61B 5/00; A61B 17/135; G06F 19/00

(52) U.S. Cl. CPC A61B 5/1107; G06F 19/3406; A61B 5/6831; A61B 5/1121; A61B 5/7278; A61B 5/0024; A61B 5/742; A61B 5/7475; A61B 5/6898; A61B 5/4082; A61B 17/135; A61B 2090/064

(57) ABSTRACT

The present invention relates to a portable devise that is able to quantify spasticity. In one aspect, the invention allows clinicians to objectively quantify spasticity in an accurate and repeatable manner. The device is designed to accommodate for different limb sizes and includes an accelerometer and a force sensing resistor to obtain quantitative data. The device further includes a data acquisition module where the data collected can be processed and sent to an output device.

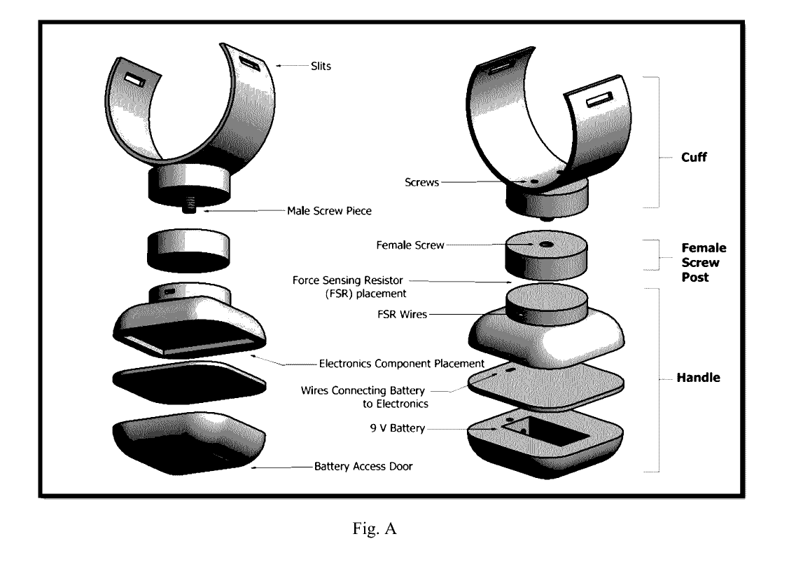

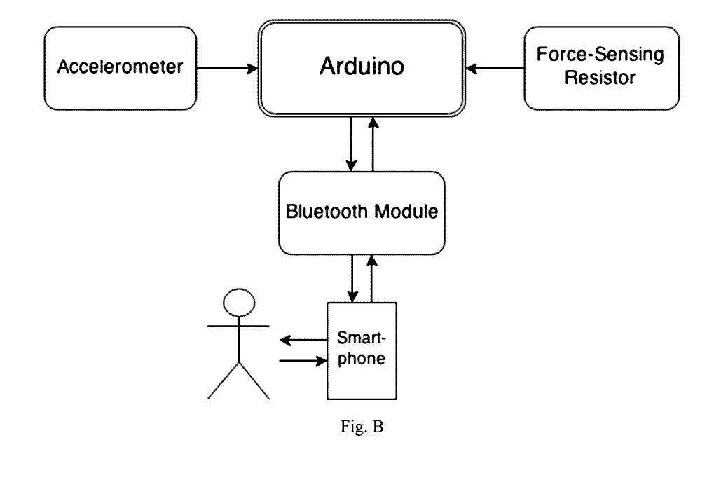

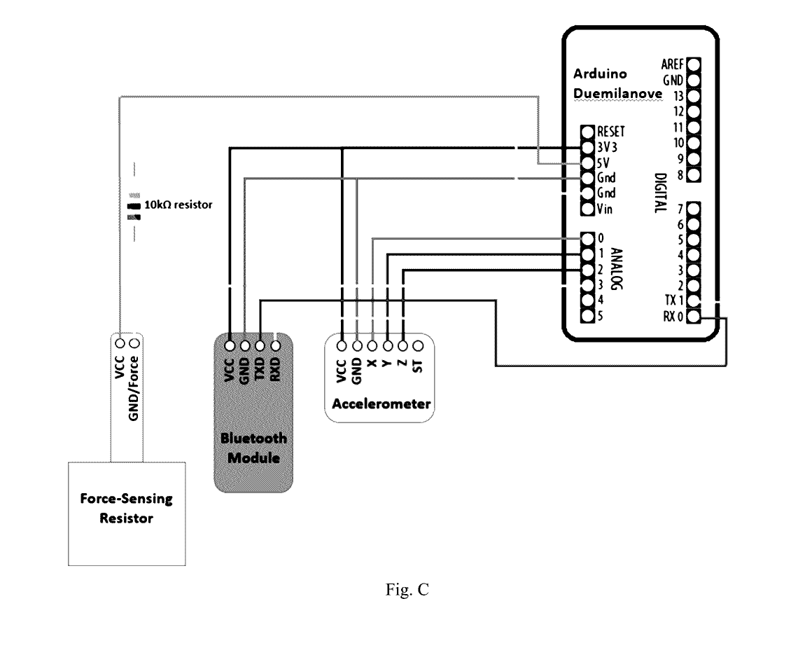

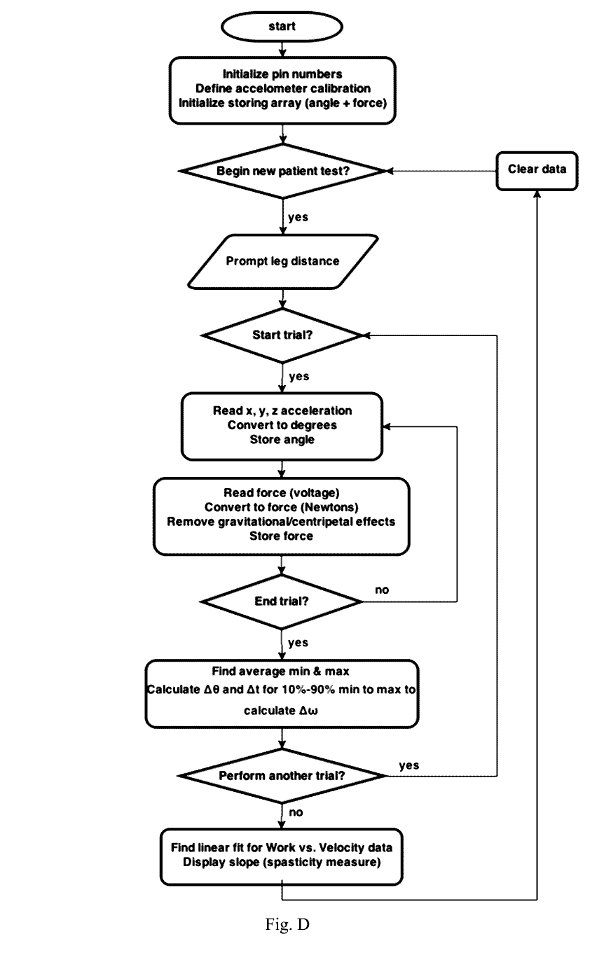

### CROSS REFERENCE TO RELATED APPLICATION

This application claims priority to and the benefit of U.S. Provisional Patent Application Ser. No. 62/155,669 filed May 1, 2015, which is incorporated by reference herein in its entirety.

### BACKGROUND

1. Field of the Invention

The present invention is a hardware and software integrated device that is able to objectively quantify muscle spasticity.

2. Description of the Related Art/Background

Muscle spasticity affects millions worldwide, and it is a prevalent symptom of people with cerebral palsy, spinal cord or brain Injury, stroke, and multiple sclerosis, Spasticity refers to a velocity-dependent resistance to passive muscle stretch caused by damage to the motor cortex. It refers to stiffness in the muscles that needs to be treated and monitored constantly for patients who have diseases which cause this symptom. The adverse effects of spasticity include inhibition of movement, difficulty speaking, and developmental problems, and can significantly affect a patient's quality of life, Treatment options exist to mitigate the amount of spasticity in cerebral palsy patients: However, many of these treatments are marred by life-threatening or debilitating side effects and only should be applied in specific cases.

There are three primary methods that exist to measure spasticity, including the Modified Ashworth scale, electromyography (EMG), and deep tendon reflex tests.

The Modified Ashworth Scale is the test most commonly used in a clinical setting. This test involves manually moving a limb through a range of motion to passively stretch muscle groups and qualitatively analyzing the degree of spasticity about the tested joint. The clinician subjectively assigns an integer to the degree of spasticity between 0 and 4 (with 4 being most spastic). This test is rapid and inexpensive, but difficult to repeat accurately as it depends purely on the judgment of the physician performing the test and may vary from test to test or from physician to physician.

The deep tendon reflex test is also subjective in nature, and it is similar to the Modified Ashworth Scale in that the response is subjectively graded from 0 to 4, instead of stretching the muscle passively, however, the muscle tendon is briskly tapped to elicit a reflex, and then the physician Judges the degree to which the muscle responds.

Electromyography (EMG) is a quantitative way of measuring spasticity, like that shown in Patent Publication number WO2006102764 A1 (includes EMG and an angle sensor to give spastic data) and WO2010121353 A1 (a portable device which combines muscle electrical activity, angular velocity and limb articulation angle to give a spasticity value). However, the amount of background noise introduced from moving the joint renders the test inaccurate. Additionally, the test is far too time consuming, expensive, and cumbersome to be feasibly applied in a clinical setting. Other ways of quantifying spasticity include a study which tried to objectively measure spasticity by applying a constant force about the moving arm and observing the velocity reduction in the movement about that joint. The amount of velocity reduction can then be translated into an index of severity of the spasticity. This study however does not take into account upper and lower ranges of spasticity and does not sample the data fast enough to provide good data.

### BRIEF SUMMARY OF THE INVENTION

Spasticity can be quantified by using data obtained from several key parameters including the velocity of motion, range of motion and the resistance to motion during joint rotation. The present invention relates to an optimal diagnostic tool to quantify spasticity in a clinical setting by measuring the three factors needed to assess spasticity: the spastic limb's range of motion, velocity of motion, and resistive force when rotated about a joint at a relatively constant velocity by a clinician. The designed device is able to compete with the Modified Ashworth Scale in ease of use, cost and exam time, and is able to characterize spasticity in a more accurate and repeatable manner.

One objective of the invention is to be able to objectively quantify spasticity by removing the clinician's subjective judgement from the quantification method.

A second objective is to accurately and precisely quantify spasticity. The high degree of accuracy of the accelerometer and force-sensing resistor, in conjunction with the robust nature of the method described, provides a spasticity value that is reproducible and far more precise than the Modified Ashworth Scale.

Another objective is to use a method that is easy to learn and take minimal time to use. The method involves moving the limb through its range of motion, a technique that is already used in the Modified Ashworth Scale and familiar to all clinicians already. The entire test itself takes less than one minute, which is comparable to the Modified Ashworth Scale, yet does not sacrifice accuracy.

Another objective is to be able to quantify spasticity for any sized limb. Different sizes of cuffs can be detached and fastened onto the device.

Still another objective is to be able to standardize the dosages of medicinal treatments for spasticity by using the collected data from this device.

### BRIEF DESCRIPTION OF THE DRAWINGS

For a better understanding of the present invention, reference may be made to the accompanying drawings in which:

Fig. A is an illustration of an exploded view of how the entire device pieces together. Notice that the handle is split into three parts to show the interior design;

Fig. B illustrates a connectivity diagram showing different components of device and how they interact. All the computations are performed by the Arduino, which is transferred to the smartphone. An alternate embodiment can include a printed circuit board (PCB) to replace the microcontroller;

Fig. C illustrates a circuit diagram connecting FSR, Bluetooth module, and accelerometer, to the Arduino. Again, an alternate embodiment can have a printed circuit board (PCB) to replace the microcontroller; and

Fig. D illustrates a software Flowchart detailing the computational analysis that is performed by the microcontroller.

### LIST OF ITEMS

[100] Cuff
[102] Slits
[104] Screws
[106] Cylindrical Post
[108] Male Screw Protrusion
[110] Cuff Arms
[200] Female Screw Post
[202] Female Screw
[204] Female Screw Post Bottom side
[300] Handle
[302] Upper handle post face
[304] Upper handle post
[306] Force Sensing Resistor (FSR) Wire Inlet
[308] Electronics component cavity
[310] Upper handle
[312] Wire Inlet
[314] Middle Handle Piece
[316] Battery
[318] Battery' Cavity
[320] Battery Access door
[322] Bottom Handle

Several drawings have been presented to illustrate features of the present invention. The scope of the present invention is not limited to what is shown in the figures.

### DETAILED DESCRIPTION OF THE PREFERRED EMBODIMENTS

The present invention relates to a hardware and software integrated system that is able to accurately quantify spasticity for patients, This new device is seamlessly integrated so that it is intuitive for the physician to use and receive reliable data.

### Hardware Components

Referring to the illustration above marked A, the preferred embodiment of this invention includes hardware that consists of three main parts, namely the cuff 100, the female screw post 200, and the handle 300.

The cuff 100 includes two bendable arms 112, and the cuff is attached using flat screws 104 onto a cylindrical post 108. There is a slit 102 on each arm to allow the removable elastic Velcro strap to tighten the cuff around the patient's limb. The bottom end of the cylindrical post 106 has a male screw protrusion 108 which allows the entire cuff to be easily attached and detached to the female screw post 200. Having different sized cuffs also help further account for any possible variation in limb sizes and can be used for quantifying spasticity in the arms and in the legs. Having different cuff sizes allows the device to fit securely around any patient's limb, ranging from the smallest wrists to the largest ankles of a patient of any age, as determined previously. In order to not require new electronics for each cuff size, the cuffs screw on and off of the device interchangeably. Furthermore, the elastic band can be removed, entirely, allowing for an appropriate range of limb sizes to be accommodated. The force sensing resistor is sandwiched between the handle 300 and the female screw post 200 and properly secured using double sided tape on both sides of the force transducer. The handle 300 is split up into three sections. The uppermost section houses the electronic components and the FSR wire inlet 306 on the upper handle post 304 allows the force sensing resistor to connect to the Arduino microcontroller inside the upper handle 310. The middle handle piece 314 is a thin flat piece to create a physical separation but between the electronics and the battery. The small wire inlet 312 on the side of this flat piece allows wiring to connect the battery to the electronics. This wire is typically sealed off, creating an air-tight, water-tight seal between the electronics and the open battery component so that minimal damage can occur to the device. Finally, the bottom handle 322 houses the battery with a battery access door 320 on the very bottom of this piece.

After the device is manufactured and assembled, the handle 300, the force sensing resistor and the female screw post 200 can be all attached to form one piece using double sided tape. The physician simply has to screw the correct sized cuff onto the female screw post 200, and the device is ready to assess the patient's spasticity. The patient's limb is strapped in the cuff 100 and secured using an elastic Velcro strap that passes through the two slits 102 completing the circle around the patient's limb and securing it. The cuff arms 110 can be made from a shape-retaining plastic that allows flexible bending to occur while maintaining a rigid shape. Once the patient's arm is secure in the cuff, the physician simply needs to engage the handle and move the patient's limb at various speeds to accurately quantify spasticity. The force sensing resistor senses the input force, while the accelerometer produces signals that result in measurement of the speed and range of motion. These three parameters are then analyzed together to acquire the final quantified spasticity value.

In some embodiments of this invention, the hardware include different shapes and sizes for the handle to allow for better ergonomics. The cylindrical post 106, the female screw post 200, and the upper handle post 304 are not limited to a cylindrical shape and can be either rectangular or rounded rectangular in shape, or any other shape. The wire inlets may change locations based off of design feasibility. The cuff arms 110 are not limited to one specific material (flexible or rigid) and can be attached onto the cylindrical post 106 in various ways such as a snap fit form of attachment or various other methods other than using screws 104.

### Software Components

The preferred embodiment collects data accurately in real time using a microcontroller, an accelerometer, and a force transducer housed within a portable chassis that is strapped to the patient's limb, and collects data over the course of joint rotations at different velocities. Using interactive software like a smartphone application to control the device, the clinician grips the handle of the device and uses it to pivot the limb at three or more different velocities. At each velocity, the force sensing resistor senses the input force, while the accelerometer measures the speed and range of motion. From these measured parameters, the torque-angle relationship can be derived and integrated to give the work applied at each velocity. Spasticity leads to a positive correlation between work and velocity.

The connectivity diagram for the software component is shown in the illustration marked B.

The main component used by the software is the Arduino Duemilanove. This is a microcontroller that collects the data from the accelerometer, FSR, and Bluetooth module at a specified times. In order to connect it to all three major components, a different wiring scheme is set up for each of them. The device is powered by an external portable battery source. All of the devices connect to the Arduino, which act as the main processor for this device. It is worth noting that all output is interpreted by the Arduino as a voltage measure, ranging from 0 to 5V. This voltage reading is expressed as a number ranging from 0 to 1023, for a total of 2¹⁰ levels. An output of 0 represents 0V, 1023 represents 5V.

The circuitry for all of the electronic parts is connected as shown in the circuit diagram in the illustration marked C. In order to allow the accelerometer to communicate with the Arduino, a total of 5 connections are made: a 3.3V power source (red), a ground (gray) to power the accelerometer, and three analog input connections (green, blue, and purple), each one collecting the proper acceleration in the x, y, and z axes. All of the acceleration values are outputted as integers between 0 and 1023. The values are calibrated by scaling the x, y, and z axis outputs to match −1g and +1g. Note that unique calibration values exist for each device. Thus, each accelerometer must undergo custom calibration to obtain these values to output accurate acceleration values. The acceleration values are then converted to angle values using simple trigonometry (inverse tangent, where the horizon is 0 degrees, with a range from −180 to 180 degrees).

To implement the force-sensing resistor (FSR), there is one connection to the 5V source (pink) and one connection to ground and analog input (orange), with a 10 kΩ resistor to ensure a baseline value of 0. Note that the resistor can be of any resistance so long as the final force calculations are scaled appropriately. The FSR itself operates by decreasing in resistance with greater force, thereby increasing the voltage output. In order to convert the output voltage reading to force values, the resistance to force calibration curve was derived from the paper as a logarithmic plot. The empirical data was separated into three linear regimes and three separate linear fits were applied, as shown in the illustration marked D.

The third component, the Bluetooth module, was connected to the Arduino in order to allow for external communication with the microcontroller. To power the device, a connection was made to the 3.3V power source (red) as well as ground (gray). In addition, two connections (light blue and dark blue) were made to transmit and receive data between the Bluetooth module and Arduino.

Once wired, the smartphone, with Bluetooth settings turned on, can connect to the Bluetooth module just as it would with any other Bluetooth device. Tap on the device name once to connect and enter the default connection code of ‘1234.’ This setup only needs to be done once for every phone. The smartphone is programmed to communicate with the Bluetooth module, sending signals of ‘0’ or ‘1’ to stop and start, respectively. A signal of ‘2’ indicates combining the data, integrating to find the work, and applying a linear fit to obtain the spasticity value. This value is then displayed on the phone application (App.) to be read by the clinician who is administering the spasticity test.

The software component, once designed and set up initially, never needs to be set up again by the user. The clinician simply switches on the device, connects their Bluetooth with one tap of a button, and begins administering the test.

In some embodiments of this invention, the inputs to the microcontroller are received by the use of tactile screens, dials, buttons or voice command, and the outputs can be displayed on a monitor, a LCD screen, tactile screens or printed on paper. Alternative methods of force measurement are within the scope of the invention such as a strain gauge or flexible stretch sensor. Power can optionally be supplied by an outlet or adapted to be rechargeable in nature.

Several descriptions and illustrations have been presented to aid in understanding the present invention. One with skill in the art will realize that numerous changes and variations may be made without departing from the spirit of the invention. Each of these changes and variations is within the scope of the present invention.

1. A portable device for measurement of spasticity in a limb, comprising:
a cuff which is secured to a patient's limb
a handle which is gripped by a physician
a data acquisition module
wherein data from angular velocity, force, and/or range of motion are received and processed before being sent to an output device.

2. The device as claimed in claim 1 wherein said data acquisition module can be a microcontroller which integrates output from an accelerometer and a force sensor or a custom designed printed circuit board which integrates output from a force sensor.

3. The device as claimed in claim 1 wherein said device is handheld and portable.

4. The device as claimed in claim 1 wherein all the electronic components are housed inside the handle of the device.

5. The device as claimed in claim 3 wherein a clinician uses said device to move a patient's limb through its full range of motion to acquire the spasticity value.

6. The device as claimed in claim 3 wherein a clinician uses said device to move a patient's limb at different velocities to acquire the spasticity value.

7. The device as claimed in claim 1 wherein the mechanism for securing the cuff to the limb is constructed to accommodate a wide range of patient limb sizes.

8. The device as claimed in claim 1 wherein elastic and VELCRO® are used to secure the limb within the cuff

9. The device as claimed in claim 6 wherein the cuff is made from semi-flexible material

10. The device as claimed in claim 6 wherein the cuff is detachable from the data acquisition module and interchangeable

11. The device as claimed in claim 1 wherein the cuff is detachable from the handle and interchangeable

12. The device as claimed in claim 2 wherein said accelerometer uses direction of proper acceleration at any given time to measure range of motion and velocity

13. The device as claimed in claim 2 wherein said force sensor can measure the force at any given time

14. The force sensor as claimed in claim 13 where the force sensor can be a force-sensing resistor or a strain gauge

15. The device as claimed in claim 2 wherein said microcontroller integrates the outputs of the accelerometer and force sensing resistor.

16. The device as claimed in claim 1 wherein said data acquisition module computes velocity-dependent change in force, thereby allowing spasticity quantification to be defined as the degree to which force changes with changing velocity

17. The device as claimed in claim 1 wherein said data acquisition module uses electronic data transfer to output data from microcontroller to viewing device

18. The device as claimed in claim 17, wherein said data transfer method is via Bluetooth technology

19. The viewing device as claimed in claim 17, wherein said viewing device is an electronic device with GUI capability or a device with an LCD

20. The device as claimed in claim 17, wherein said transferred data is received by a smartphone or computer or a device with an LCD

21. The viewing device as claimed in claim 20, wherein said smartphone, computer or a device with an LCD has an application to receive and output data

22. The device as claimed in claim 1 wherein said data acquisition module uses electronic data transfer to allow microcontroller to be controlled by user interface

23. The device as claimed in claim 22, wherein the user interface for controlling is located on the same device.

24. A device as claimed in claim 1 wherein output is displayed on a monitor, smartphone, or printed on paper

25. A device as claimed in claim 1 wherein the control module is an electronic device with an LCD screen, a smartphone or a personal computer such as desktop or laptop computer

26. A device as claimed in claim 1 further comprising a battery to deliver electrical power required for functioning

27. (canceled)

28. (canceled)

29. (canceled)

30. (canceled)
